(19) United States
(12) Patent Application Publication
(10) Pub. No.: US 2016/0184200 A1
(43) Pub. Date: Jun. 30, 2016

(54) UV-CURABLE NAIL COATING FORMULATIONS BASED ON RENEWABLE ACIDS, LACTONES CYCLIC ETHER, LACTAMS

(71) Applicant: MYCONE DENTAL SUPPLY COMPANY, INC., Cherry Hill, NJ (US)

(72) Inventor: George LEIN, Elkton, MD (US)

(21) Appl. No.: 14/910,772

(22) PCT Filed: Aug. 7, 2014
(86) PCT No.: PCT/US14/50068

§ 371 Date: Feb. 8, 2016

Related U.S. Application Data
(60) Provisional application No. 61/863,370, filed on Aug. 7, 2013.

(51) Int. Cl. A61K 8/36; A61K 8/87; A61K 8/37; A61Q 3/02; A61K 8/55

(52) U.S. Cl. CPC A61K 8/36; A61Q 3/02; A61K 8/55; A61K 8/375; A61K 8/87; A61K 2800/81; A61K 2800/95

(57) ABSTRACT

A photopolymerizable composition for forming a cosmetic coating for natural and artificial nails of humans and animals comprising a photoinitiator and a polyurethane prepared by reacting a polyisocyanate with an acid, lactone, cyclic polyether, amine, or lactam that has been prepared from a renewable resource with a (meth)acrylate monomer and decorative, cosmetic finger-nail and toenail coatings formed by curing such compositions under UV or other light radiation.

### CROSS-REFERENCE TO RELATED APPLICATIONS

Benefit of U.S. provisional application Ser. No. 61/863,370, filed Aug. 7, 2013, is claimed.

### BACKGROUND OF THE INVENTION

This invention relates to compositions for photopolymerizable coatings forming cosmetic films that are especially useful for human and animal nail coatings. Such compositions are capable of free radical addition curing reactions with unsaturated ethylenic pendant groups on compounds upon exposure to actinic radiation in the presence of a photoinitiator.

Ultra-violet radiation (UV) is the most conventional form of actinic radiation used to cure gels in this art, however, visible light curing systems are also known. Professional nail technicians most typically apply UV curable gels designed for coating and/or sculpting nails. Such UV-curable gels are usually composed of acrylic or methacrylic monomers and oligomers in a gel-like state that requires curing under a UV lamp. Such nail finishes can be applied directly to natural fingernails or toenails, or alternatively can be applied to nail extensions bonded to fingernails. Multiple coatings can also be used. There has been a strong movement to design environmentally friendly chemical compositions. In the field of photopolymerizable nail coating compositions, there have been no commercially available compositions which are marketed as being environmentally friendly.

US Patent publication No. 2011-0182837 and U.S. Pat. No. 5,985,951 describe the use of materials based on renewable polyols which improve the environmental footprint of the compositions. These polyols can be converted to polyesters or polyurethanes to make suitable materials for radiation curable nail coatings.

Neither of the above publications disclosed the use of acids, cyclic ethers, lactones, amines, or lactams based on renewable resources as materials which can be converted to suitable reactive urethanes. Such materials for use in radiation curable nail coatings are not known. WO/2012130601 discloses the use of acids derived from renewable resources for the formation of reactive polyesters which may be used in radiation curable nail coatings. However, this application specifically teaches that reactive urethanes are not part of the formulations.

It is an object of this invention to provide nail coating compositions which have properties which are either comparable to or superior to existing commercial radiation curable nail coating compositions and are more environmentally friendly than conventional nail coating compositions.

### SUMMARY OF THE INVENTION

This object and others which will become apparent from the following disclosure are achieved by the present invention which comprises in one aspect a photopolymerizable composition for forming a cosmetic coating for natural and artificial nails of humans and animals, the composition comprising a photoinitiator and a reactive urethane derived from an acid, lactone, cyclic polyether, amine, and/or lactam that has been prepared from a renewable resource.

In one aspect of the invention, the reactive urethane is prepared from a polyester polyol wherein said polyester polyol is prepared from at least one compound containing two or more acid or ester groups, said compound having been derived from a renewable resource.

In a preferred aspect of the invention, the reactive urethane is prepared from a polyester polyol wherein said polyester polyol is prepared from at least one compound containing one or more acid or ester groups and at least one polyol wherein both the acid or ester containing compound and the polyol have been derived from a renewable resource.

The polyester polyol can be prepared from at least one cyclic lactone derived from a renewable resource.

In some embodiments the polyester polyol can be prepared from the at least one cyclic lactone and a polyol wherein both the cyclic lactone and the polyol are derived from a renewable resource.

The reactive urethane can be prepared from a compound containing a polyol made from a cyclic ether that has been derived from a renewable resource.

In some embodiments the reactive urethane can be prepared from a polyester polyol wherein said polyester polyol is prepared from at least one compound containing two or more acid or ester groups and at least one polyol wherein both the acid or ester containing compound and the polyol have been derived from a renewable resource and the polyol has been derived from a cyclic ether that has been derived from a renewable resource.

The reactive urethane can be prepared from a polyester polyol which has been prepared from compounds containing at least one hydroxyl group and at least one acidic group, said compounds having been derived from renewable sources.

In some embodiments the reactive urethane can be prepared from a polyester polyol which has been prepared from compounds containing at least one hydroxyl and at least one acidic group and a polyol wherein both the compound containing acidic and hydroxyl groups and the polyol are derived from renewable resources.

The reactive urethane can be prepared from a polyamino polyamide wherein said polyamino polyamide is prepared from at least one compound containing two or more acid or ester groups, said compound having been derived from a renewable resource.

In some embodiments the reactive urethane can be prepared from a polyamino polyamide wherein said polyamino polyamide is prepared from at least one compound containing two or more acid or ester groups and at least one polyamine wherein both the acid or ester containing compound and the polyamine have been derived from a renewable resource.

The reactive urethane can be prepared from a polyamino polyamide, said polyamino polyamide having been prepared from a cyclic lactam derived from a renewable resource.

The reactive urethane can be prepared from a polyamino polyamide, said polyamine polyamide having been prepared from a cyclic lactam and a polyamine wherein both the cyclic lactam and the polyamine have been derived from a renewable resource.

The reactive urethane can be prepared from a polyamino polyamide, said polyamine polyamide having been prepared from a compound containing at least one amino group and at least one acidic group wherein the compound is derived from renewable resources.

The reactive urethane can be prepared from a polyamino polyamide, said polyamino polyamide having been prepared from a compound containing at least one amino group and at least one acid or ester group and a polyamine wherein both the compound containing at least one amino group and at least one acid or ester group and the polyamine have been derived from a renewable resource.

As is customary in this radiation curable nail coating art, the composition may include one or more optional additives. The optional additives can be monomers, polymers, oligomers, crosslinkers, pigments, colorants, dyes, UV-absorbing or reflecting materials, micas, glitters, flavors, fragrances, thixotropic additives, dispersants and/or any other additives known in the art of radiation curable nail coatings.

When applied to human or artificial nails, the compositions cure under UV radiation or natural light and form a hard coating which adheres to the nail. The compositions can optionally be designed to be easily removed with solvent. Urethanes are widely used for the production of nail gels due to the broad range of properties which they can impart.

The reactive urethane can contribute to improved application performance, adhesion, wear, and/or durability of photopolymerized nail coatings.

The compositions, methods of use, and resultant artificial nails or cured coatings are also aspects of this invention.

### DETAILED DESCRIPTION

The use of materials derived from renewable resources is advantageous in that many resources are being depleted by their use to form various petrochemical based materials. By renewable we mean materials that are derived from plants, animals, bacteria, fungi, algae and other sustainable sources which can be produced in shorter time frames than is required for the formation of petroleum or coal. These materials often give an improved “carbon footprint” compared to materials derived from petroleum or coal based sources. In addition, these materials can be renewed since they do not require long periods of time to form and can be generated as needed from various sources. Suitable materials made from renewable resources are prepared via processes including but not limited to fermentation, bacterial digestion, enzymatic reactions, pyrolysis, and other chemical reactions wherein, after processing, at least 50% of the carbon atoms contained in the material are derived from plants, animals, or other living matter such that the living matter was a living organism less than 10 years prior to processing. Direct production by living organisms followed by extraction of the material either after the death of the organism or by extraction without destruction of the organism to yield either the final material or further materials for processing is suitable.

Some materials from sustainable resources have been available historically and many new ones are under development today and others can be expected to become available in the future. To date, however, reactive urethanes derived from sustainable resources have not been applied to the field of radiation curable nail coatings.

These reactive urethanes can be prepared by methods known in the art, for example from reactions of polyester polyols or polyamino polyamides with diisocyanates and hydroxyl containing monomers. Methods including reaction of a polyester polyol or a polyamino polyamide with a diisocyanate followed by reaction with a hydroxyl containing monomer or by reaction of a hydroxyl containing monomer with a diisocyanate followed by reaction with a polyester polyol or polyamino polyamide are commonly used.

Examples of isocyanates useful in the invention include isophorone diisocyanate, hexamethylene diisocyanate, trimethyl hexamethylene diisocyanate, 4,4′-methylene dicyclohexyl diisocyanate, toluene diisocyanate, methylene diphenyl diisocyanate, polymeric methylene diphenyl diisocyanate, tetramethylxylylene diisocyanate, triisocyanurate, isocyanatoethyl methacrylate, isophorone diisocyanate trimer, hexamethylenediisocyanate trimer, hexamethylene diisocyanate biuret, and hexamethylene diisocyanate uretdione. Isocyanate terminated prepolymers prepared from polyester, polyether or other hydroxyl functional materials may also be used. Mixtures of materials containing isocyanate groups may also be used.

Reaction of polyamido polyamines with isocyanato ethyl methacrylate yields a polyurea which also is defined as a polyurethane for the purposes of this invention.

Examples of the hydroxyl-containing monomers include 2-hydroxyethyl acrylate, 2-hydroxyethyl methacrylate, diethylene glycol monoacrylate, diethylene glycol monomethacrylate, glycerol (meth)acrylate, glycerol di(meth)acrylate, sorbitol (meth)acrylate, di(meth)acrylate and tri(meth)acrylate, 2-hydroxypropyl acrylate, 2-hydroxypropyl methacrylate, 3-hydroxypropyl acrylate, 3-hydroxypropyl methacrylate, tetraethylene glycol mono(meth)acrylate, pentaethylene glycol mono(meth)acrylate, dipropylene glycol monomethacrylate, and dipropylene glycol monoacrylate, dipentaerythritol penta acrylate, dipentaerythritol penta methacrylate, pentaerythritol triacrylate, pentaerytritol trimethacrylate, caprolactone (meth)acrylates, polycaprolactone (meth)acrylates, polyethyleneoxide mono(meth)acrylates, polypropyleneoxide (meth)acrylates, ditrimethyol propane tetra(meth)acrylate, carbohydrate based (meth)acrylic monomers, and hydroxyl alkyl (meth)acrylamides such as n-methylol acrylamide. The most preferred hydroxyl-containing monomers are hydroxyethyl methacrylate (HEMA) and hydroxypropyl methacrylate (HPMA). Mixtures of more than one hydroxyl-containing monomer can be used. These hydroxyl containing monomers may be used to prepare the polyurethanes and/or used as a component of the curable composition. These monomers are preferably prepared from acrylic or methacrylic acid which has been derived from a renewable resource and most preferably prepared from acrylic or methacrylic acid and other components wherein both the acid and the reactive component are derived from a renewable resource.

The polyester polyols can be derived from various known methods in the art. For example condensation polymerization of polyacids or polyesters with polyols leads to polyester polyols and the ratio of polyacid or polyester to polyol can be adjusted to give the desired molecular weight. Various polyacids which are obtained from natural and renewable resources are available commercially today and others are under development. Polyacids based on renewable resources that are available commercially or in development include succinic acid, sebacic acid, azeleic acid, 2,5 furan dicarboxylic acid, fumaric acid, malonic acid, aconitic acid, C₁₁-C₁₆ diacids being developed by Cathay Industrial Biotech, oxalic acid, Pripol dimer fatty acids available from Croda, itaconic acid, Empol diacids, and adipic acid. Polyacids which do not contain alkene functionality are preferred over those which do contain alkene functionality. In addition polyacids with less than four acid functional groups are preferred over those containing four or more four functional acid groups. Polyacids containing less than 11 carbon atoms are preferred since removal of cured composition with typical solvents used for removal of the cured composition, such as acetone, is more difficult when long chain acids are used. Diacids containing no alkene functionality and less than 11 carbon atoms are most preferred.

The polyester polyols can be prepared via condensation polymerization of hydroxyl containing acids and polyols. Examples of hydroxyl containing acids which are commercially available or in development which are derived from sustainable sources include lactic acid, glycolic acid, 3-hydroxy propionic acid and citric acid. Preferred compounds contain no alkene functionality one hydroxyl unit and one carboxylic unit and less than 11 carbon atoms.

The polyester polyols can be formed by the ring opening polymerization of cyclic lactones. Examples of these materials which are available commercially or under development include lactide, caprolactone, 3-hydroxy butyrolactone, valerolactone, beta-propiolactone, butyrolactone, angelilactones, butenyl lactone. Preferred lactones contain less than 11 carbon atoms and no alkene functionality. These polyester polyols may be prepared using polyols to catalyse the ring opening in polymerization. Preferably the polyols are derived from renewable resources. An example the synthesis of suitable diols from lactide can be found in Colloid Polym Sci (2009) 287:671-681

Examples of polyols prepared from cyclic ethers include those made by ring opening polymerization of ethylene oxide or propylene oxide which have been derived from renewable resources.

The reactive urethanes are preferably prepared from polyester polyols derived from a renewable resource. These polyols may be used in condensation reactions with the polyacids or to initiate the ring opening polymerizations of lactones or in condensation reactions with compounds containing acid and hydroxyl functionality. Examples of commercially available polyols and those under development include 1,3 propane diol, 1, 4 butanediol, 1,2 propane diol, ethylene glycol, sucrose, glucose, fructose, resorcinol, xylitol, glycerol, arabinitol, 2,5 dihydroxymethyl tetrahydrofuran, 2,5 dihydoxymethyl furan, 2-amino 1, 4 butanediol, 1,5 pentane diol, 2-methyl 1,4 butanediol, 1,4 pentane diol, propylene glycol, glycerol, ethylene glycol, isosorbide, and Pripol dimer diols. Preferred polyols do not contain alkene functionality and have less than four hydroxyl groups. Most preferred are diols containing no alkene functionality.

Polyamino polyamides may be prepared by ring opening polymerization or of cyclic lactams with polyamines Examples of cyclic lactams which are available or are being developed from renewable resources include caprolactam, 2-pyrrolidone and n-methyl pyrrolidone.

These polyamides are preferably made using materials derived from renewable resources such as 1,4 diamino butane, 2,5 bis(aminomethyl) tetrahydrofuran and 2-methyl 1,4 butane diamine.

Alternatively polyamino polyamides may be prepared by condensation polymerization of the polyacids or polyesters derived from renewable resources with polyamines Preferably these polyamino polyamides may be prepared using polyamines derived from renewable resources.

Polyamino polyamides may also be prepared from the condensation polymerization of compounds containing amino groups and acid groups in the presence of polyamines Representative examples of these materials which can be derived from renewable resources include glycine and alanine. The polyamine is preferably also derived from renewable resources.

The photopolymerizable compositions of the invention form a cosmetic coating for natural and artificial nails of humans and animals. The compositions comprise one or more photopolymerizable urethanes derived from an acid, lactone, cyclic polyether, amine, or lactam that has been prepared from renewable resource. Preferably the reactive urethanes contain (meth)acrylate functionality.

The urethane oligomer can be cured by exposure to UV or visible radiation, as is conventional in this art. In some embodiments the composition further comprises an ethylenically unsaturated cross-linking monomer. The preferred cross-linking monomers are (meth)acrylate crosslinking monomers. The composition can include other monomers, oligomers, and/or polymers which are ethylenically unsaturated and which react in the presence of radiation.

The compositions may also comprise (meth)acrylate monomers and/or oligomers prepared by reacting a core polyol with a (meth)acrylate monomer and optionally one or more co-reactants selected from the group consisting of an organic diisocyanate, a polyacid, polyester, cyclic lactone, cyclic lactam, ethylene oxide, propylene oxide, epoxy compounds, polyols, and polyamines, wherein the core polyol is derived from a renewable resource. These photopolymerizable monomers and/or oligomers can in some embodiments comprise an ethylenically-unsaturated crosslinking reagent. Such crosslinking reagent can be a (meth)acrylate monomer or any other ethylenically unsaturated monomer. In some embodiments the (meth)acrylate monomer has one polyol unit and the (meth)acrylate oligomer has multiple polyol units.

In addition to the above-described (meth)acrylate-based polymerizable monomers, crosslinkers, oligomers and urethanes, other polymerizable monomers, oligomers or polymers of monomers which contain at least one free radical polymerizable group in the molecule may be used. These materials may be derived from renewable resources or from more traditional sources such petroleum or coal feedstocks.

These monomers and oligomers may contain other groups such as carboxyl groups to improve adhesion. Examples of monomers and oligomers which contain carboxyl groups that can be used to improve adhesion include (meth)acrylic acid, Sarbox resins available from Sartomer, Inc., Glycine, N-2-hydroxy-3-(2-methyl-1-oxo-2-propenyl)oxypropyl-N-(4-methylphenyl) acid, ethylene glycol ethyl phosphate, and the reaction products of hydroxyl containing (meth)acrylates with anhydrides such as succinic anhydride, trimellitic anhydride, maleic anhydride and phthalic anhydride.

Examples of optional monomers are esters and amides of acrylic and methacrylic acid. The esters of acrylic and methacrylic acid are herein termed (meth)acrylic ester. Specific but not limiting examples of mono methyl (meth)acrylic esters include: methyl (meth)acrylate, ethyl (meth)acrylate hydroxypropyl (meth)acrylate, ethyl (meth)acrylate, butyl (meth)acrylate, hydroxy ethyl (meth)acrylate, butoxyethyl (meth)acrylate, diethylaminoethyl (meth)acrylate, 2-ethylhexyl (meth)acrylate, ethoxyethyl (meth)acrylate, t-butyl aminoethyl (meth)acrylate, methoxyethylene glycol (meth)acrylate, phosphoethyl (meth)acrylate, methoxy propyl (meth)acrylate, methoxy polyethylene glycol(meth)acrylate, phenoxyethylene glycol (meth)acrylate, phenoxypolyethylene glycol (meth)acrylate, 2-hydroxy-3-phenoxypropyl (meth)acrylate, 2-(meth)acryloxyethylsuccinic acid, 2-(meth)acryloylethylphthalic acid, 2-(meth)acryloyloxypropylphthalic acid, stearyl (meth)acrylate, isobornyl (meth)acrylate, 3-chloro-2-hydroxypropyl (meth)acrylate, tetrahydrofurfuryl (meth)acrylate, (meth)acrylamides and allyl monomers.

Optional oligomers include urethane(meth)acrylates having at least two or more acryl or methacryl groups and a urethane group. Examples include urethanes based on aliphatic, aromatic, polyester, and polyether polyols and aliphatic, aromatic, polyester, and polyether diisocyanates capped with (meth)acrylate end-groups. Isocyanate prepolymers can also be used in place of the polyol-diisocyanate core.

Other oligomers include epoxy (meth)acrylates and epoxy urethane (meth)acrylates having at least two or more acryl or methacryl groups and, optionally, a urethane group. Examples include epoxy (meth)acrylates based on aliphatic or aromatic epoxy prepolymers capped with (meth)acrylate end-groups. An aliphatic or aromatic urethane spacer can be optionally inserted between the epoxy and the (meth)acrylate end group(s). Other oligomers include acrylated polyester oligomers having at least two or more acryl or methacryl groups and a polyester core. Acrylated polyether oligomers having at least two or more acryl or methacryl groups and a polyether core are also optional. Acrylated acrylate oligomers having at least two or more acryl or methacryl groups and a polyacrylic core can be used in some embodiments.

These reactive urethanes, epoxies, polyesters, polyethers and acrylics are available from several suppliers including BASF Corporation, Bayer MaterialScience, Bomar Specialties Co., Cognis Corporation, Cytec Industries Inc., DSM NeoResins, Eternal Chemical Co, Ltd., IGM Resins, Rahn AG, Sartomer USA, LLC, Double Bond Chemical, Miwon and SI Group, Inc.

Examples of crosslinkers are difunctional methacryl esters such as 1,4-butane diol di(meth)acrylate, 1,6-hexanediol di(meth)acrylate, 1,9-nonanediol di(meth)acrylate, 1,10-decanediol di(meth)acrylate, neopentyl glycol di(meth)acrylate, 2-methyl-1,8-octane diol di(meth)acrylate, glycerol di(meth)acrylate, ethylene glycol di(meth)acrylate, triethylene glycol di(meth)acrylate, polyethylene glycol di(meth)acrylate, polypropylene glycol di(meth)acrylate, ethoxylated propylene glycol di(meth)acrylate, ethoxylated polypropylene glycol di(meth)acrylate, polyethoxypropoxy di(meth)acrylate, ethoxylated bisphenol A di(meth)acrylate, propoxylated bisphenol A di(meth)acrylate, propoxylated ethoxylated bisphenol A di(meth)acrylate, bisphenol-A glycidyl methacrylate, tricyclodecanedimethanol di(meth)acrylates glycerin di(meth)acrylate, ethoxylated glycerin di(meth)acrylate, bis acrylamides, bis allyl ethers and allyl (meth)acrylates.

Examples of tri and or higher (meth)acryloyl esters crosslinking agents include trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol penta(meth)acrylate, dipentaerythritol hexa(meth)acrylate, and ethoxlated iscyanuric acid tri(meth)acrylates.

The photopolymerizable monomers or oligomers can be urethanes based on aliphatic, aromatic, polyester, and polyether polyols as well as the core polyol, and aliphatic, aromatic, polyester, and polyether diisocyanates capped with (meth)acrylate end-groups. Isocyanate prepolymers can also be used in place of the polyol-diisocyanate core. Epoxy (meth)acrylates and epoxy urethane (meth)acrylates which have at least two or more acryl or methacryl groups and, optionally, a urethane group can be used, for example epoxy (meth)acrylates based on aliphatic or aromatic epoxy prepolymers capped with (meth)acrylate end-groups. An aliphatic or aromatic urethane spacer can be optionally inserted between the epoxy and the (meth)acrylate end group(s). Acrylated polyester oligomers, useful in the present invention, have at least two or more acryl or methacryl groups and a polyester core. Suitable acrylated polyether oligomers have at least two or more acryl or methacryl groups and a polyether core. Suitable acrylated acrylate oligomers have at least two or more acryl or methacryl groups and a polyacrylic core. These reactive urethanes, epoxies, polyesters, polyethers and acrylics are available from several suppliers including, for example, BASF Corporation, Bayer MaterialScience, Bomar Specialties Co, Cognis Corporation, Cytec Industries Inc., DSM NeoResins, Eternal Chemical Co, Ltd, IGM Resins, Rahn AG, Sartomer USA, LLC, and SI Group, Inc.

Combinations of two or more materials containing free radical polymerizable groups may be used).

Examples of suitable photoinitiators are benzyl ketones, monomeric hydroxyl ketones, polymeric hydroxyl ketones, alpha-amino ketones, acyl phosphine oxides, metallocenes, benzophenone, benzophenone derivatives, and the like. Specific examples include: 1-hydroxy-cyclohexylphenylketone, benzophenone, 2-benzyl-2-(dimethylamino)-1-(4-(4-morphorlinyl)phenyl)-1-butanone, 2-methyl-1-(4-methylthio)phenyl-2-(4-morphorlinyl)-1-propanone, diphenyl-(2,4,6-trimethylbenzoyl) phosphine oxide, phenyl bis(2,4,6-trimethylbenzoyl) phosphine oxide, benzyl-dimethylketal, isopropylthioxanthone, and mixtures thereof.

Photo accelerators such as aliphatic or aromatic amines may also be included in the gel as well as fillers, inhibitors, plasticizers and adhesion promoters.

Suitable colorants which can be incorporated into the color concentrates include barium, calcium and aluminum lakes, iron oxides, chromates, molybdates, cadmiums, metallic or mixed metallic oxides, talcs, carmine, titanium dioxide, chromium hydroxides, ferric ferrocyanide, ultramarines, titanium dioxide coated mica platelets, and/or bismuth oxychlorides. Preferred pigments include D&C Black No. 2, D&C Black No. 3, FD&C Blue No. 1, D&C Blue No. 4, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4., D&C Red No. 6, D&C Red No. 7, D&C Red No. 17, D&C Red No. 21, D&C Red No. 22, D&C Red No. 27, D&C Red No. 28, D&C Red No. 30. D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, as well as others listed on the FDA color additives website, and Annex IV of the Cosmetic Directive 76/768/EEC, Coloring Agents Permitted in Cosmetics as of Mar. 1, 2010.

Pigment levels in the composition can be from greater than 0.1-wt % up to as much as 20-wt %. Colored pigments are preferred from 0.5 up to 10-wt %. Mixtures of TiO2 and colored pigments are most preferred.

Preferably the compositions comprise a colorant which is conventional in photocurable nail coatings.

Upon exposure to actinic radiation the photopolymerizable composition polymerizes to form a hard coating on the nails. In some embodiments the reactive urethane contributes to improved adhesion, application performance, wear, and/or durability of photopolymerized nail coatings.

The coating compositions can contain solvents, pigments, modifying resins, plasticizers, and other compounds mixed and maintained in a liquid solution.

### EXAMPLES

The following examples illustrate a few embodiments of the invention.

### Example 1

### Preparation of Polyester Oligomer from 1000 Mw Polyethylene Glycol Succinate

To a resin kettle equipped with a stirrer was charged, under dry air, 0.2 moles of isophorone diisocyanate (IPDI) and 0.15 g of dibutyl tin dilaurate (DBDTL) and 0.5 g of butylated hydoxy toluene (BHT). Then 0.2 moles of hydroxyethyl acrylate (HEA) were added over 1 hr. The reaction exothermed to 65° C. and 0.1 moles of a 1000 Mw ethylene glycol succinate which had been prepared from bio-succinic acid derived from renewable cellulosic resources, namely corn, (Myriant DG-110 brand) was added. The reaction was held at 85° C. until no isocyanate peak remained in the infrared spectrum.

### Example 2

### Preparation of Polyester Oligomer from 1750 Mw Polypropylene Glycol Succinate

The procedure of Example 1 was used using 0.3 moles of IPDI, 0.23 g DBTDL, 0.8 g BHT, 0.3 moles HEA, and 0.15 moles of a 1750 Mw polypropylene glycol succinate which had been prepared from bio-succinic acid derived from renewable source. The polyol was an experimental product, Desmogreen, received from Bayer Material Science and the bio-succinic acid was sourced from Bioamber.

### Example 3

### Preparation of Polyester Oligomer from 500 Mw Polypropylene Glycol Succinate

The procedure of Example 1 was used using 0.6 moles of IPDI, 0.2 g DBTDL, 0.8 g BHT, 0.6 moles HEA, and 0.3 moles of a 500 Mw polypropylene glycol succinate which had been prepared from bio-succinic acid derived from a renewable source. The polypropylene glycol succinate was an experimental product, Desmogreen, received from Bayer Material Science and the bio-succinic acid was sourced from Bioamber.

### Example 4

### Curable Gels

UV curable nail gels were prepared using the formulations in Table 1. A 10 mil drawdown of each gel was cured for three minutes using a standard UV lamp used for curing nail gels. All gels gave acceptable properties including gloss, flexibility and adhesion to glass.

TABLE 1

| | Formula- | Formula- | Formula- |
|---|---|---|---|
| | tion 1 | tion 2 | tion 3 |
| Ingredients | % | % | % |
| HEMA¹ | 40 | 40 | 40 |
| TPO² | 5 | 5 | 5 |
| Oligomer from Example 1 | 55 | | |
| Oligomer from Example 2 | | 55 | |
| Oligomer from Example 3 | | | 55 |
| Total | 100 | 100 | 100 |

¹Hydroxyethyl methacrylate
²Diphenyl(2,4,6-trimethylbenzoyl)-Phosphine Oxide

The present invention, therefore, is well adapted to carry out the objects and attain the ends and advantages mentioned, as well as others inherent therein. While the invention has been depicted and described and is defined by reference to particular preferred embodiments of the invention, such references do not imply a limitation on the invention, and no such limitation is to be inferred. The invention is capable of considerable modification, alteration and equivalents in form and function, as will occur to those ordinarily skilled in the pertinent arts. The depicted and described preferred embodiments of the invention are exemplary only and are not exhaustive of the scope of the invention. Consequently, the invention is intended to be limited only by the spirit and scope of the appended claims, giving full cognizance to equivalents in all respects.

1. A method for preparing a photopolymerizable composition for forming a cosmetic coating for natural and artificial nails of humans and animals comprising a photoinitiator and a photopolymerizable reactive urethane derived from preparing an acid, lactone, cyclic polyether, amine, or lactam that has been prepared from a renewable resource, preparing a reactive urethane from a composition comprising the acid, lactone, cyclic polyether, amine, or lactam prepared from the renewable resource, combining the reactive urethane with a photoinitiator to form the cosmetic coating composition, applying the cosmetic coating composition onto natural nails of humans or animals, and reacting the cosmetic coating composition under ultraviolet light to form a cosmetic coating on the nails.

2. The method of claim 1 wherein the acid, lactone, cyclic polyether, amine, or lactam prepared from a renewable resources is prepared by a process which comprises a step selected from the group consisting of fermentation, bacterial digestion, enzymatic reactions, and pyrolysis wherein, after the step at least 50% of the carbon atoms contained in the acid, lactone, cyclic polyether, amine, or lactam are derived from plants, animals, or other living matter.

3. The method of claim 2 wherein the plants, animals, or other living matter was a living organism less than 10 years prior to the step selected from the group consisting of fermentation, bacterial digestion, enzymatic reactions, and pyrolysis.

4. The method of claim 1 wherein the acid, lactone, cyclic polyether, amine, or lactam prepared from a renewable resources is prepared by a process which comprises direct production by living organisms followed by extraction of the material either after the death of the organism or by extraction without destruction of the organism to yield the acid, lactone, cyclic polyether, amine, or lactam.

5. (canceled)

6. (canceled)

7. (canceled)

8. (canceled)

9. (canceled)

10. (canceled)

11. (canceled)

12. (canceled)

13. The method of claim 1 wherein the acid, lactone, cyclic polyether, amine, or lactam that has been prepared from a renewable resource contributes to improved application performance, adhesion, wear, and/or durability of photopolymerized nail coatings.

14. (canceled)

15. (canceled)

16. (canceled)

17. (canceled)

18. (canceled)

19. (canceled)

20. (canceled)

21. (canceled)

22. (canceled)
